(19) United States
(12) Patent Application Publication
(10) Pub. No.: US 2023/0313108 A1
(43) Pub. Date: Oct. 5, 2023

(54) Sampling Device And Cell Culture System

(71) Applicant: Terumo Kabushiki Kaisha, Tokyo (JP)

(72) Inventor: Masatsugu IGARASHI, Kanagawa (JP)

(21) Appl. No.: 18/206,464

(22) Filed: Jun. 6, 2023

Related U.S. Application Data
(63) Continuation of application No. PCT/JP2022/008731, filed on Mar. 2, 2022.

(30) Foreign Application Priority Data
Mar. 3, 2021 (JP) 2021-033629

(51) Int. Cl. C12M 1/00

(52) U.S. Cl. CPC C12M 29/10; C12M 23/34

(57) ABSTRACT

A cell culturing system includes a culturing device including a plurality of reactors, and a sampling device that receives a liquid sample from the culturing device. A sample introduction channel fluidically connects the cell culturing device and the sampling device. The sample introduce channel includes a temporary storage unit that can temporarily store a sample. A sample in each of the plurality of reactors may flows into the temporary storage unit under the operation of a pump. A combined sample may be obtained by combining the plurality of samples received from the plurality of reactors. The temporary storage unit sends the combined sample to a sampling channel of the sampling device.

### CROSS-REFERENCE TO RELATED APPLICATIONS

The present application is a continuation application of the International Patent Application No. PCT/JP2022/008731 filed on Mar. 2, 2022, which designated the U.S. and claims the benefit of priority from Japanese Patent Application No. JP2021-033629 filed on Mar. 3, 2021. The entire disclosures of the above-identified applications are incorporated herein by reference.

### FIELD

The present disclosure relates to a sampling device for collecting a liquid sample from a cell culturing device and to a cell culturing system that includes the sampling device and the cell culturing device.

### BACKGROUND

A sampling device for collecting a liquid sample from a culturing device. The sampling device includes a sampling channel and an introduction pump that is configured to draw a sample from a sample introduction channel that is connected to the culturing device into the sampling channel. The sampling device may also include a detection unit downstream of the sampling channel. The detection unit is configured to detect components contained in the sample and amounts (e.g., concentrations) of the components.

The culturing device may include a plurality of reactors as culture vessels in order to improve the efficiency of cell culture. The culturing device can culture cells in each of the plurality of reactors, for example, by seeding cells on the reactor and supplying a culture medium to the reactor.

Each of the plurality of reactors may have different culture states as the result of, for example, differences in flowing state of the culture medium. Therefore, when a sampling device is connected to the culturing device and is configured to detect a sample flowing out from an unspecified reactor, the culture state of each reactor cannot be accurately grasped.

If the sampling device is instead configured to detect a sample for each of the plurality of reactors, the number of times of sampling and the sample amount greatly increase, which may decrease the sampling efficiency.

Accordingly, there is a need for a sampling device and a cell culturing system capable of more efficiently detecting a sample for each of a plurality of reactors.

### SUMMARY

The present disclosure provides a sampling device for collecting a liquid sample from a culturing device. The culturing device may have a plurality of reactors for culturing a cell on the basis of a flow of a culture medium. the sampling device may include a sampling channel through which the sample flows, a detection unit provided in the sampling channel, and a sample introduction channel that connects the sampling channel and a culturing device. The sample introduction channel may be connected to the sampling channel upstream of the detection unit. The sampling device may also include a pump that is configured to move the sample through the sample introduction channel and a control unit that operates the pump. The sampling device may also include a temporary storage unit provided in the sample introduction channel and capable of temporarily storing the sample. The temporary storage unit may sequentially receive a sample for each of the plurality of reactors under the operation of the pump and may store a combined sample including at least a portion of each of the received samples from the different reactors. The temporary storage may be configured to send the combined sample to the sampling channel.

The present disclosure provides a cell culturing system for collecting a liquid sample from a culturing unit. The culturing unit may include a plurality of reactors for culturing a cell on the basis of a flow of a culture medium. The cell culturing unit may be configured to sequentially supply the culture medium to the plurality of reactors. The cell culturing system may include a sampling channel through which the sample flows, a detection unit provided in the sampling channel, and a sample introduction channel that connects the sampling channel and the culturing unit. The sample introduction channel may connect to the sampling channel upstream of the detection unit. The cell culturing system may also include a pump that allows the sample to flow through the sample introduction channel and a control unit that operates the pump. The cell culturing system may also include a temporary storage unit provided in the sample introduction channel and capable of temporarily storing the sample. The temporary storage unit may be configured to sequentially receive a sample for each of the plurality of reactors under the operation of the pump and to store a combined sample including at least a portion of each of the received samples from the different reactors. The temporary storage may be configured to send the combined sample to the sampling channel.

The sampling device and the cell culturing system can efficiently detect a sample for each of a plurality of reactors.

### BRIEF DESCRIPTION OF DRAWINGS

FIG. 1 is a perspective view of a cell culturing system in accordance with at least one example embodiment of the present disclosure.

FIG. 2 is a schematic illustrating a channel for a culture medium in the cell culturing system illustrated in FIG. 1 in accordance with at least one example embodiment of the present disclosure.

FIG. 3 is a schematic illustrating a channel configured to receive samples from a plurality of reactors in accordance with at least one example embodiment of the present disclosure.

FIG. 4 is a schematic illustrating a channel of an example sampling device, for example, for use with the cell culturing system illustrated in FIG. 1 in accordance with at least one example embodiment of the present disclosure.

FIG. 5 is a flowchart illustrating a sampling method as performed by the sampling device illustrated in FIG. 4 in accordance with at least one example embodiment of the present disclosure.

FIG. 6 is a flowchart illustrating a sampling step of the sampling method illustrated in FIG. 5 in accordance with at least one example embodiment of the present disclosure.

FIG. 7 is a schematic illustrating an operations of a temporary storage step in accordance with at least one example embodiment of the present disclosure.

FIG. 8 is a schematic illustrating operations of a combined sample outflow step in accordance with at least one example embodiment of the present disclosure.

FIG. 9 is a schematic illustrating a channel of another example sampling device, for example, for use with the cell culturing system illustrated in FIG. 1 in accordance with at least one example embodiment of the present disclosure.

FIG. 10A is a flowchart illustrating a sampling step performed by the sampling device illustrated in FIG. 9 in accordance with at least one example embodiment of the present disclosure.

FIG. 10B is a flowchart illustrating a culturing step in accordance with at least one example embodiment of the present disclosure.

FIG. 11 is a schematic illustrating a channel of another example sampling device, for example, for use with the cell culturing system illustrated in FIG. 1 in accordance with at least one example embodiment of the present disclosure.

### DETAILED DESCRIPTION

Preferred embodiments of the present disclosure will now be described in detail with reference to the drawings.

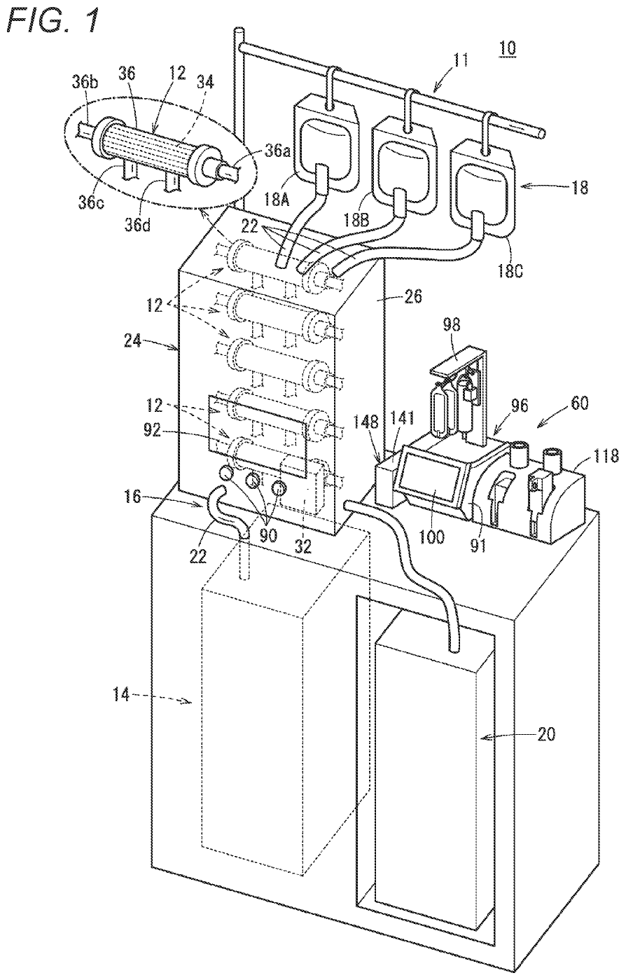

A cell culturing system 10 for culturing biological cells in regenerative medicine is illustrated in FIG. 1. A sampling device 60 may be used with the cell culturing system 10. The sampling device 60 may be configured to sample a culture medium during culture of the cells by the cell culturing system 10 and to measures the state of the culture medium. For example, the cell culturing system 10 may continue cell culture for a long period of time by discharging lactic acid, carbon dioxide, and/or the like (including, for example, unused medium and oxygen) as generated during cell culture from a reactor 12, which may be a cell culture vessel, while supplying a culture medium or oxygen to the reactor 12.

The biological cells are not particularly limited. In at least one example embodiment, the biological cells may include cells contained in blood (e.g., T cells and/or the like) and/or stem cells (e.g., ES cells, iPS cells, mesenchymal stem cells, and/or the like). Any appropriate culture medium may be selected for use with the selected biological cells. For example, the culture medium may include a basic solution that includes amino acids, vitamins, serum, and/or the like. The basic solution may include, for example, a balanced salt solution (BSS).

The cell culturing system 10 may include a culturing device 11 (also referred to as a culturing unit) in which multiple reactors 12 are set and cells are actually cultured and a sampling device 60 (also referred to as a sampling unit) that collects a liquid sample from the culturing device 11 during the culture. The cell culturing system 10 may be configured such that a culture medium flows through and is cultured in each of the multiple reactors 12 allowing several times as many cells as cultured in one reactor 12 to be obtained without greatly changing the culturing period. Although FIG. 1 illustrates the culturing device 11 as including five reactors 12, it should be appreciated that the number of reactors 12 as provided in the culturing device 11 is not particularly limited. For example, in certain embodiments, the number of reactors 12 as provided in the culturing device 11 may exceed five, and in other embodiments, the number of reactors 12 as provided in the culturing device 11 may be less than five. Although not illustrated, it should also be appreciated that in at least one example embodiment, the cell culturing system 10 may have a configuration in which a plurality of culturing devices 11 may be connected to one sampling device 60. Further still, although the c the culturing unit and the sampling unit are separately illustrated and described, it should be appreciated that in at least one example embodiment, the culturing unit and the sampling unit are integrated (unified).

The culturing device 11 may include a culture medium reservoir 14 configured to store a culture medium, a flow channel 16 provided between the reactor 12 and the culture medium reservoir 14, a plurality of medical bags 18 connected to the flow channel 16, and a waste liquid unit 20 configured to store a liquid discharged through the flow channel 16.

The culture medium reservoir 14 may include a hard tank that is configured to store a large amount of culture medium. The flow channel 16 may include multiple tubes 22. The multiple tubes 22 may be connected to the multiple reactors 12, the culture medium reservoir 14, the plurality of medical bags 18, and/or the waste liquid unit 20, respectively.

The plurality of medical bags 18 may include a cell solution bag 18A configured to store a liquid (e.g. cell solution) that includes cells, a cleaning solution bag 18B configured to store a cleaning solution, and/or a stripping solution bag 18C configured to store a stripping solution. Although not illustrated, it should be appreciated that, in at least one example embodiment, the plurality of medical bags 18 may include a collection bag (not illustrated) configured to receive cultured cells. The cleaning solution may include a liquid that can be used for priming the reactor 12 and/or the flow channel 16. The cleaning solution may include a buffer solution and/or a physiological saline solution. The buffer solution may include, for example, phosphate buffered salts (PBS) and/or tris-buffered saline (TBS). The stripping solution is a liquid for stripping the cells cultured by a culture treatment. The stripping solution may include, for example, trypsin and/or EDTA solution.

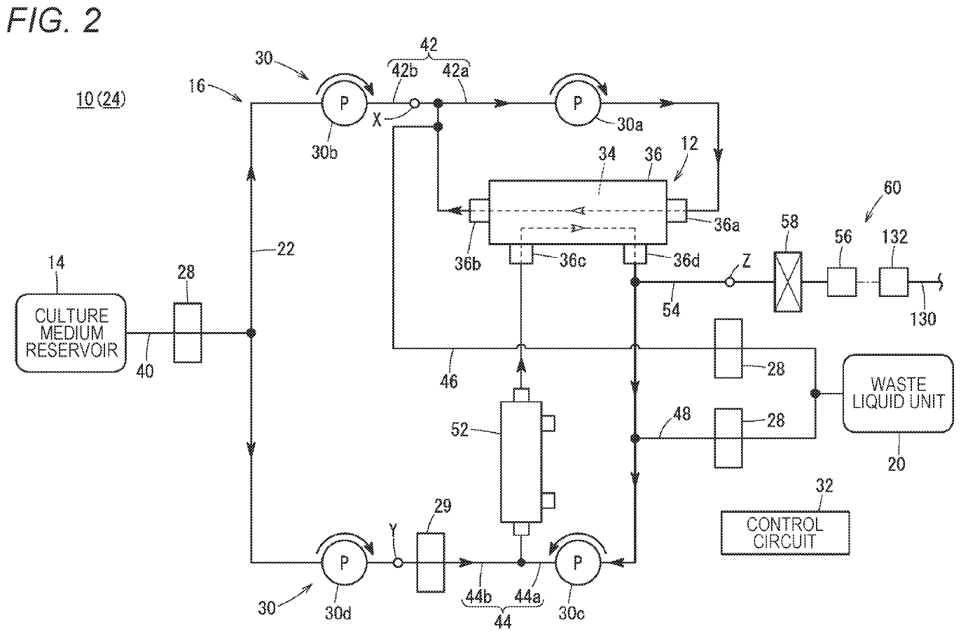

When the cell culturing system 10 is constructed, the flow channel 16 may be set so as to pass through a flow path control mechanism 24 of the culturing device 11. The flow path control mechanism 24 may include a housing 26 that is configured to house a part of the flow channel 16. The flow path control mechanism 24 may also include a clamp 28 that is configured to open and close a predetermined tube 22, a pump 30 that is configured to allow a liquid in the tube 22 to flow, and a control circuit 32 that is configured to control operations of the clamp 28 and/or the pump 30 (see FIG. 2). The claim 28, the pump 30, and the control circuit 32 may be disposed in the housing 26.

The multiple reactors 12 may be accommodated in the housing 26 of the flow path control mechanism 24. The reactor 12 may include a plurality of (10,000 or more, for example) hollow fibers 34 and a case 36 that is configured to accommodate the plurality of hollow fibers 34. Although not illustrated, it should be appreciated that, in at least one example embodiment, each of the hollow fibers 34 may have a lumen, and cells may be seeded an inner peripheral surfaces defining the lumens. Although not illustrated, it should be appreciated that, in at least one example embodiment, each of the hollow fibers 34 may have a plurality of pores that allows communication between the outside of the hollow fiber 34 and the lumen. For example, each pore may transmit a solution or lower molecular weight substance without transmitting larger molecular weight substances, like cells or proteins. A culture medium or the like may be supplied to the cells seeded on the inner peripheral surface of the hollow fiber 34 through the lumen or the pores. Hereinafter, the configuration in which the liquid mainly flows through the lumen of the hollow fiber 34 may be referred to as intra capillary (IC), and the configuration in which the liquid mainly flows through the outer side of the hollow fiber 34 may be referred to as extra capillary (EC).

Each of the cases 36 may include a first IC terminal 36a and a second IC terminal 36b each of which communicates with the lumens of the hollow fibers 34, and a first EC terminal 36c and a second EC terminal 36d each of which communicates with a space outside the hollow fibers 34 in the case 36. The tube 22 may be connected to each terminal.

The configurations of the flow channel 16 between one reactor 12 and the culture medium reservoir 14 and the flow path control mechanism 24 will be specifically described, by way of example, below with reference to FIG. 2. The flow channel 16 may include a medium delivery route 40 that may be connected to the culture medium reservoir 14, and an IC route 42 (internal route) and an EC route 44 (external route) that may branch from the medium delivery route 40. The IC route 42 may include a channel for supplying a liquid to the lumen of the hollow fibers 34. The EC route 44 may include a path for supplying liquid into the case 36 outside the hollow fibers 34.

The IC route 42 may include an IC circulation circuit 42a capable of circulating liquid with the reactor 12, and an IC supply circuit 42b through which liquid can flow from the culture medium delivery route 40 to the IC circulation circuit 42a. The IC circulation circuit 42a may be connected to the first IC terminal 36a and the second IC terminal 36b of the reactor 12 and may include an IC circulation pump 30a that is configured to allow liquid to flow through the lumen of the hollow fibers 34. An IC waste liquid circuit 46 that is configured to discharges a culture medium to the waste liquid unit 20 may be connected to the IC circulation circuit 42a downstream of the reactor 12. The IC supply circuit 42b may be provided with an IC supply pump 30b configured to allow liquid to flow from the culture medium delivery route 40 to the IC circulation circuit 42a.

The EC route 44 may include an EC circulation circuit 44a capable of circulating liquid with the reactor 12, and an EC supply circuit 44b through which liquid can flow from the culture medium delivery route 40 to the EC circulation circuit 44a. The EC circulation circuit 44a may be connected to the first EC terminal 36c and the second EC terminal 36d of the reactor 12 and may include an EC circulation pump 30c that is configured to circulate liquid on the outside of the hollow fibers 34. A gas exchanger 52 may be provided upstream of the reactor 12 in the EC circulation circuit 44a. The gas exchanger 52 may be configured to discharge carbon dioxide mixed in the culture medium and to mix a predetermined gas component (for example, nitrogen N₂: 75%, oxygen O₂: 20%, carbon dioxide CO₂: 5%) with the culture medium. An EC waste liquid circuit 48 may be configured to discharge a culture medium to the waste liquid unit 20 and may be connected to the EC circulation circuit 44a downstream of the reactor 12. The EC supply circuit 44b may include an EC supply pump 30d configured to allow liquid to flow from the culture medium delivery route 40 to the EC circulation circuit 44a.

Although not illustrated, it should be appreciated that, in at least one example embodiment, a plurality of medical bags 18 (e.g., cell solution bag 18A, cleaning solution bag 18B, and/or stripping solution bag 18C) may be connected to the IC supply circuit 42b upstream side of the IC supply pump 30b or to the EC supply circuit 44b upstream side of the EC supply pump 30d via a plurality of tubes 22 in addition to the culture medium reservoir 14. In at least one example embodiment, the medical bags 18 may be replaced with a collection bag and/or the like using, for example, a sterile connecting device that sterilizes and bonds the bag depending on the intended use.

The sampling device 60 may be connected to the EC circulation circuit 44a of the culturing device 11 at a position (between the reactor 12 and the EC waste liquid circuit 48) near the downstream side (second EC terminal 36d) of the reactor 12. One end of a sample outflow channel 54 through which a culture medium as a liquid sample flows out may be connected to the EC circulation circuit 44a. A culturing-device-side connector 56 may be provided at the other end of the sample outflow channel 54. The culturing-device-side connector 56 may be mutually connectable to a sampling-device-side connector 132 of the sampling device 60. The sampling device 60 may be connected to the IC circulation circuit 42a on the downstream side (second IC terminal 36b) of the reactor 12 via the sample outflow channel 54.

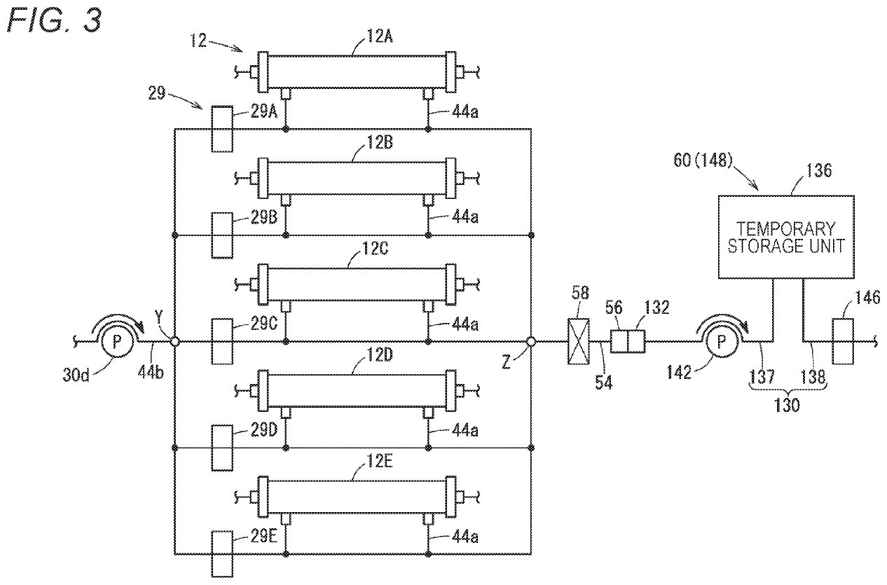

As illustrated in FIGS. 2 and 3, the cell culturing system 10 may include multiple IC circulation circuits 42a and multiple EC circulation circuits 44a corresponding to the multiple (five) reactors 12. That is, another IC circulation circuit 42a and another EC circulation circuit 44a (not illustrated) that circulate liquid to another reactor 12 may be connected in parallel to a branch point X between the IC supply pump 30b and the IC circulation circuit 42a and a branch point Y between the EC supply pump 30d and the EC circulation circuit 44a. The EC supply circuit 44b between the branch point Y and each EC circulation circuit 44a may be provided with a supply clamp 29 configured to switch between supply and stop of the supply of the culture medium to the corresponding EC circulation circuit 44a.

The five reactors 12 in FIG. 3 may be referred to as reactors 12A to 12E below in order from the top to the bottom. The supply clamps 29 provided in the EC supply circuits 44b may be referred to as supply clamps 29A to 29E corresponding to the reactors 12A to 12E, respectively. The culturing device 11 may open any one of the supply clamps 29A to 29E and closes the other four clamps while rotating the EC supply pump 30d. As a result, the culture medium may be supplied to the EC circulation circuit 44a in which the supply clamp 29 is opened, and the culture medium may flow through the reactor 12 along with the circulation in the EC circulation circuit 44a. Although not illustrated, it should be appreciated, that in at least one example embodiment, the culturing device 11 may include multiple IC circulation circuits 42a corresponding to the reactors 12A to 12E. Although not illustrated, it should be appreciated that, in at least one example embodiment, the IC supply circuit 42b connected to each of the multiple IC circulation circuits 42a may include a supply clamp for selectively allowing the culture medium to flow.

In order to be connected to each of the multiple reactors 12, the sample outflow channel 54 may branch at a branch point Z and may be connected to each EC circulation circuit 44a. An aseptic filter 58 may be provided in the sample outflow channel 54 on the culturing-device-side connector 56 side with respect to the branch point Z. The aseptic filter 58 may maintain an aseptic state of the culture medium circulating through the culturing device 11 (EC circulation circuit 44a).

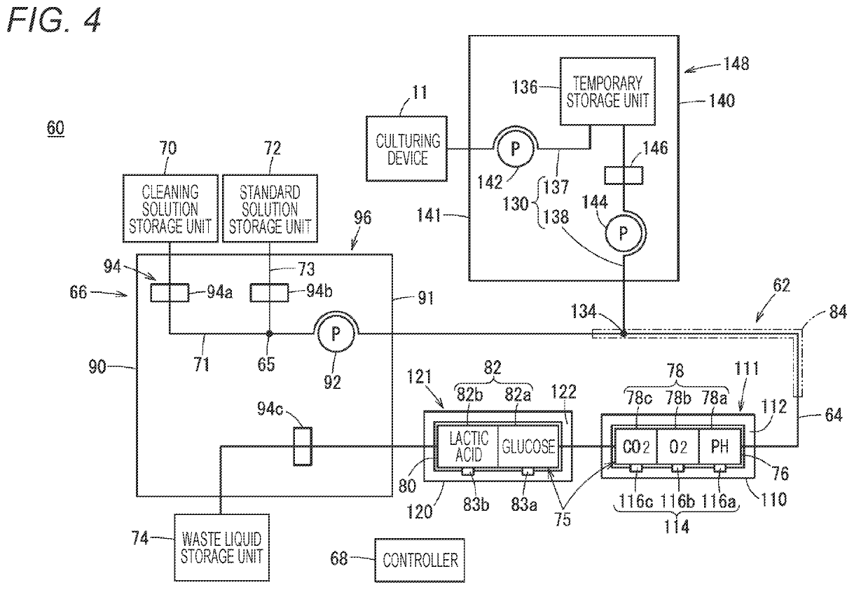

Next, a configuration of the sampling device 60 will be described with reference to FIG. 4. The sampling device 60 may be configured to collect a sample of a culture medium from one or more culturing devices 11, and to detect components contained in the sample and amounts (concentrations) of the components. The sampling device 60 may include a sampling kit 62 having a sampling channel 64 through which a sample may be collected, a plurality of mechanisms 66 in which the sampling kit 62 may be detachably set, and a controller 68 that may be configured to control operations of the plurality of mechanisms 66. In at least one example embodiment, the sampling kit 62 may be a disposable product that is used and thrown away. In at least one example embodiment, the plurality of mechanisms 66 is a reusable product that can be reused.

In addition to the sampling channel 64, the sampling kit 62 may include a cleaning solution storage unit 70, a standard solution storage unit 72, a waste liquid storage unit 74, and/or a detection unit 75. The detection unit 75 may include a first detection unit 76 and a second detection unit 80. The sampling channel 64 may include a flexible tube having an appropriate thickness by which the sample can pass therethrough. The cleaning solution storage unit 70 may be connected to a branch point 65 to which one end of the sampling channel 64 is connected via a cleaning solution branch path 71, and the standard solution storage unit 72 may be connected to the branch point 65 via a standard solution branch path 73. The other end of the sampling channel 64 may be connected to the waste liquid storage unit 74.

The cleaning solution storage unit 70 and the standard solution storage unit 72 may include, for example, a soft resin material formed into a bag shape (medical bag). The soft resin material may include polyvinyl chloride and/or polyolefin. The cleaning solution storage unit 70 and the standard solution storage unit 72 are not particularly limited as long as they can store liquid. The waste liquid storage unit 74 may share a tank of the waste liquid unit 20 of the culturing device 11, but it is not limited thereto, and a medical bag or the like may be applied.

The cleaning solution storage unit 70 may be configured to store a cleaning solution. The cleaning solution is not particularly limited, and may include, for example, a buffer solution, a physiological saline solution, and/or the like as described as the cleaning solution in the cleaning solution bag 18B of the culturing device 11.

The standard solution storage unit 72 may be configured to store a standard solution. The standard solution may include a liquid for calibrating the first detection unit 76 and the second detection unit 80 and may have a pH value, a glucose value (glucose concentration), and/or a lactic acid value (lactic acid concentration) set to prescribed values.

The first detection unit 76 and the second detection unit 80 may be provided in series and separated from each other at an intermediate position of the sampling channel 64. It should be appreciated that the detection unit 75 is not limited to be divided into the first detection unit 76 and the second detection unit 80 For example, in at least one example embodiment, the detection unit 75 may have a structure in which the first detection unit 76 and the second detection unit 80 are integrated, or a structure divided into three or more units.

The first detection unit 76 may include a tubular member having multiple first elements 78 that may come in contact with (in liquid contact with) the sample in a flow path in the sampling channel 64. The multiple first elements 78 may include, for example, a pH chip 78a for measuring the pH in the sample, an O₂ chip 78b for measuring the O₂ concentration in the sample, and/or a CO₂ chip 78c for measuring the CO₂ concentration in the sample. The pH chip 78a may be colored by reaction with H⁺ and OH⁻. The O₂ chip 78b may be colored by reaction with O₂. The CO₂ chip 78c may be colored by reaction with CO₂.

The second detection unit 80 may include a tubular member having multiple second elements 82 that may come in contact with (in liquid contact with) the sample in the flow path in the sampling channel 64. The second detection unit 80 may be provided downstream (waste liquid storage unit 74 side) with respect to the first detection unit 76. The multiple second elements 82 may include, for example, biosensors that react an enzyme with a circulating sample and detect a current change or the like. For example, the multiple second elements 82 may include a glucose chip 82a that is configured to measure the glucose concentration in the sample and/or a lactic acid chip 82b that is configured to measure the lactic acid concentration in the sample. The glucose chip 82a may be electrically connected to a glucose terminal 83a protruding to the outside of the tubular member. The lactic acid chip 82b may be electrically connected to a lactic acid terminal 83b protruding to the outside of the tubular member.

In addition, the sampling kit 62 may include a connection part 84 to which one or more sample introduction channels 130 may be connected between the branch point 65 of the sampling channel 64 and the first detection unit 76. The connection part 84 may be, for example, a member obtained by integrally molding a plurality of branch ports each having a valve (not illustrated) that closes when the sample introduction channel 130 is not attached and opens as the sample introduction channel 130 is attached (in FIG. 4, the connection part 84 is indicated as a range surrounded by a two-dot chain line for convenience). Alternatively, a port to which the sample introduction channel 130 can be connected with the sampling channel 64 being kept aseptic may be applied to the connection part 84.

At least a part of the sampling kit 62 may be set in a main mechanism 90 which is one of the plurality of mechanisms 66 as illustrated in FIG. 4. The main mechanism 90 may include, in the housing 91, a main-mechanism-side pump 92 and/or a plurality of clamps 94 that are configured to open and close flow paths in the respective channels (tubes). Although not illustrated, it should be appreciated that, in at least one example embodiment, the controller 68 that controls the sampling device 60 may be provided in the main mechanism 90. The sampling kit 62 may be set in the main mechanism 90, by which a main unit 96 of the sampling device 60 may be constructed.

The sampling channel 64 may extend between the branch point 65 and the connection part 84 may be disposed in the main-mechanism-side pump 92. The main-mechanism-side pump 92 may include a circular wound portion around which the sampling channel 64 can be wound so as to wrap around and rotates so as to apply a peristaltic action on the wrapping sampling channel 64 (tube), thereby allowing an internal fluid (liquid, air, etc.) to flow.

The multiple clamps 94 may include a cleaning solution clamp 94a configured to open and close the cleaning solution branch path 71, a standard solution clamp 94b configured to open and close the standard solution branch path 73, and/or a waste liquid clamp 94c configured to open and close the sampling channel 64 between the second detection unit 80 and the waste liquid storage unit 74.

The first detection unit 76 of the sampling kit 62 may be set in a first measuring instrument 110 which is one of the plurality of mechanisms 66, whereby a first sensor unit 111 may be constructed. The first measuring instrument 110 may include a holder 112 that is configured to accommodate the first detection unit 76 and a measurement body 114 fixed to the holder 112 and configured to optically measuring the plurality of first elements 78.

The measurement body 114 may include a pH detector 116a, a O₂ detector 116b, and/or a CO₂ detector 116c that may be arranged to respectively face the pH chip 78a, the O₂ chip 78b, and the CO₂ chip 78c with the first detection unit 76 being held by the holder 112. Under the control of the controller 68, the measurement body 114 may emit measurement light having a wavelength corresponding to the characteristics of each of the first elements 78, receive excitation light generated from the first element 78 by excitation, and/or transmit a detection signal thereof to the controller 68.

Further, the second detection unit 80 of the sampling kit 62 may be set in a second measuring instrument 120 which is one of the plurality of mechanisms 66, whereby a second sensor unit 121 may be constructed. The second measuring instrument 120 may include a case 122 that can accommodate the second detection unit 80 and an enzyme detector (not illustrated) electrically connected to the glucose terminal 83a and/or the lactic acid terminal 83b. The enzyme detector may be configured to detect a current value from each of the glucose chip 82a and/or the lactic acid chip 82b and to transmit a detection signal based on the current value to the controller 68.

In order to introduce a sample to be measured by the first sensor unit 111 and/or the second sensor unit 121, the sample introduction channel 130 may be connected to the connection part 84 of the sampling kit 62 (sampling channel 64). Similar to the sampling channel 64, the sample introduction channel 130 may include a flexible tube having an appropriate thickness by which the sample can pass therethrough.

The sample introduction channel 130 may include, at one end, the sampling-device-side connector 132 to be connected to the culturing-device-side connector 56 (see also FIGS. 2 and 3). Although not illustrated, it should be appreciated that, in at least one example embodiment, a plug attachable to and detachable from the connection part 84 may be provided at the other end of the sample introduction channel 130. Hereinafter, a portion where the sample introduction channel 130 may be connected to the sampling channel 64 may be referred to as a connection point 134.

A temporary storage unit 136 may be provided in the sample introduction channel 130 between the sampling-device-side connector 132 and the plug (connection point 134). The temporary storage unit 136 may be configured to temporarily store the sample flowing out of the culturing device 11 and to send the sample to the sampling channel 64. For example, the temporary storage unit 136 may include a medical bag that may be softer than the sample introduction channel 130 and that may be hung on the stand 98 fixed on the housing 91 of the main unit 96. The temporary storage unit 136 may include a hard container.

The sample introduction channel 130 may include an upstream line 137 provided between the sampling-device-side connector 132 and the temporary storage unit 136 and a downstream line 138 provided between the temporary storage unit 136 and the plug. In a state where the temporary storage unit 136 is hung on the stand 98, the upstream line 137 and the downstream line 138 may be connected to the lower side of the temporary storage unit 136 in the direction of gravity.

A part of the sample introduction channel 130 may be set in the introduction mechanism 140 which is one of the plurality of mechanisms 66, by which an introduction unit 148 of the sampling device 60 is constructed. The introduction mechanism 140 may include an upstream pump 142, an introduction pump 144, and/or a downstream clamp 146 in a housing 141. Although not illustrated, it should be appreciated that in at least one example embodiment, the introduction mechanism 140 may also include a sensor that is configured to detect pressure and air bubbles in the flow path of the sample introduction channel 130.

The introduction unit 148 may be configured such that a part of the sample introduction channel 130, the upstream pump 142, the introduction pump 144, and/or the downstream clamp 146 may be integrally handled. The sample introduction channel 130 (downstream line 138) shortly extending from the introduction unit 148 may be connected to the connection part 84 on the housing 91.

The upstream pump 142 may be disposed on the upstream line 137 (that is, between the culturing device 11 and the temporary storage unit 136) in the introduction unit 148. The introduction pump 144 and the downstream clamp 146 may be disposed on the downstream line 138 (that is, between the sampling channel 64 and the temporary storage unit 136) in the introduction unit 148. The upstream pump 142 and the introduction pump 144 may each include a circular wound portion around which the sample introduction channel 130 can be wound and rotates so as to apply a peristaltic action on the wrapping sample introduction channel 130 (tube), thereby allowing an internal fluid to flow. The downstream clamp 146 may be configured to switch between allowing the sample to flow to the sampling channel 64 from the temporary storage unit 136 and interrupting the flow by opening and closing the downstream line 138.

The controller 68 (control unit) may include a computer that includes one or more processors (not illustrated), a memory, an input/output interface, and/or an electronic circuit. The controller 68 may be configured to control the entire sampling device 60, for example, by the processor executing programs stored in the memory. In at least one example embodiment, the controller 68 may be configured to communicate information with the control circuit 32 of the culturing device 11 and to perform ganged control of the culturing device 11 and the sampling device 60. The controller 68 may be a control device integrated with the control circuit 32 of the culturing device 11.

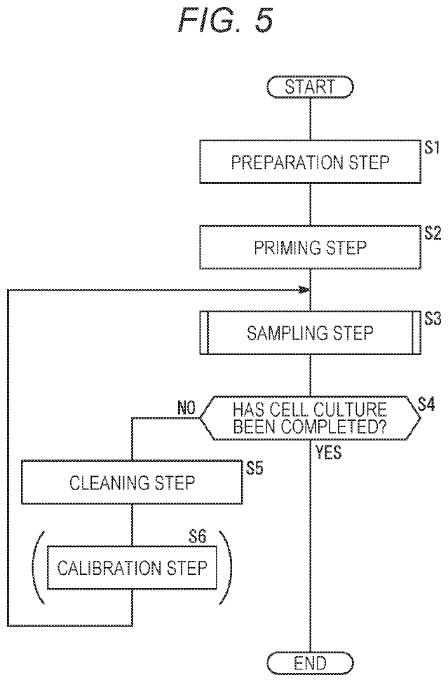

A sampling method performed, for example, by the sampling device 60 is described below with reference to FIG. 5. The sampling method may include a preparation step, a priming step, a sampling step, a cleaning step, and/or a calibration step, which can be sequentially performed.

In the preparation step (step S1), the user of the cell culturing system 10 may set (e.g. attach) the sampling kit 62 to the main mechanism 90 to form the main unit 96 as illustrated in FIG. 4. Thereafter, the user may set the first detection unit 76 exposed from the housing 91 in the first measuring instrument 110 to construct the first sensor unit 111 and may set the second detection unit 80 which are similarly exposed in the second measuring instrument 120 to construct the second sensor unit 121. The first sensor unit 111 and the second sensor unit 121 may be suspended from the stand 98.

The user may also set the sample introduction channel 130 in the introduction mechanism 140 to form the introduction unit 148. Thereafter, the user may connect the sampling-device-side connector 132 of the sample introduction channel 130 exposed from the introduction unit 148 to the culturing-device-side connector 56 and may connect the plug of the sample introduction channel 130 to the connection part 84.

In the priming step (step S2 in FIG. 5), the controller 68 may cause the cleaning solution clamp 94a and the waste liquid clamp 94c to open, may cause the standard solution clamp 94b to close, and may cause the main-mechanism-side pump 92 to rotate. Thus, the cleaning solution in the cleaning solution storage unit 70 may pass through the first detection unit 76 and the second detection unit 80 and may be discharged to the waste liquid storage unit 74.

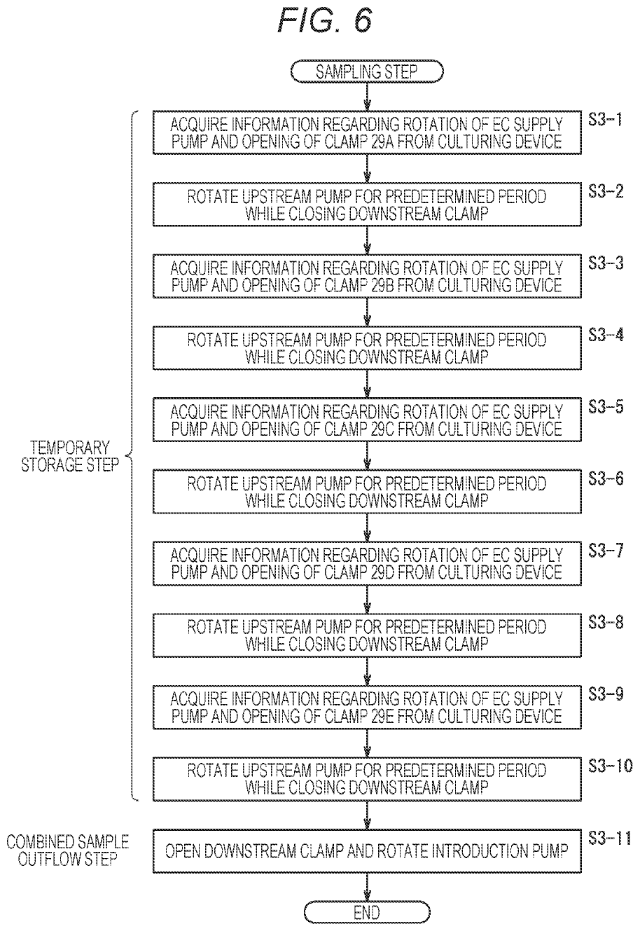

The controller 68 may cause the sample to be introduced to the detection unit 75 from the culturing device 11 in the sampling step (step S3 in FIG. 5). The controller 68 may sequentially performs a temporary storage step and a combined sample outflow step as illustrated in FIG. 6.

The temporary storage step may include collectively storing the samples that flow out of the reactors 12A to 12E in the temporary storage unit 136. In at least one example embodiment, the controller 68 may be configured to acquire information regarding the rotation of the EC supply pump 30d of the culturing device 11 and the opening of each of the supply clamps 29A to 29E and may be configured to cause the upstream pump 142 to rotate while each of the supply clamps 29A to 29E is opened. The upstream pump 142 may be configured to operate at the same rotation speed and for the same time (predetermined period) during opening of each of the supply clamps 29A to 29E. As a result, the samples from the reactors 12A to 12E may be stored in the same amount in the temporary storage unit 136.

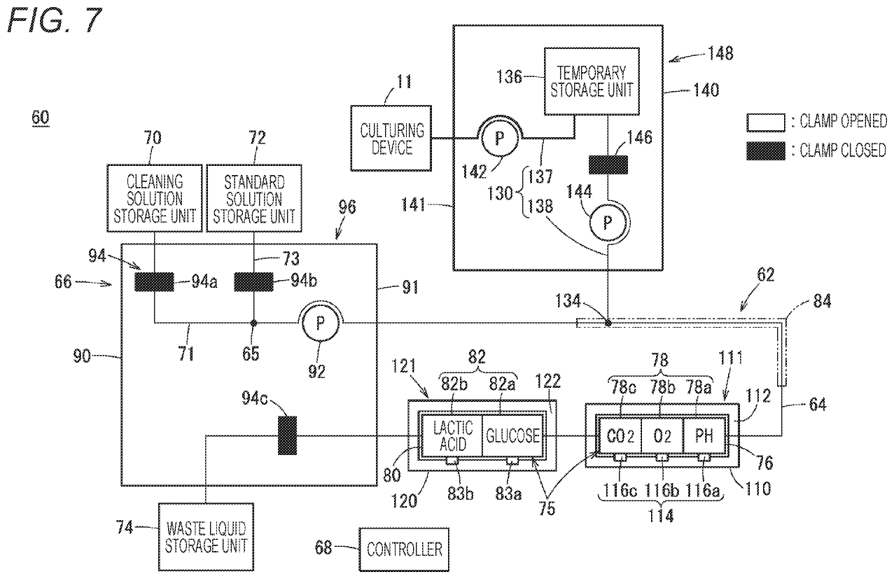

After the start of the temporary storage step, the controller 68 may be configured to acquire information regarding the rotation of the EC supply pump 30d and the opening of the supply clamp 29A from the control circuit 32 of the culturing device 11 (step S3-1). Thereafter, the controller 68 may be configured to cause the upstream pump 142 to rotate for a predetermined period while closing the downstream clamp 146 (step S3-2). At this time, the controller 68 may be configured to cause the operation of the introduction pump 144 to stop (the same applies below until the end of the temporary storage step). Thus, the sample flowing out of the reactor 12A may be stored in the temporary storage unit 136 via the upstream line 137 as illustrated in FIG. 7.

The controller 68 may be configured to rotate the upstream pump 142 to allow the sample to flow at a flow rate of about 10 mL/min, for example. The predetermined period during which the upstream pump 142 is operated may be set to about 5 seconds to about 15 seconds, although the predetermined period may depends on the opening period of the supply clamp 29A. As a result, when, for example, the upstream pump 142 is rotated for about 6 seconds, about 1 mL of the sample from the reactor 12A may be stored in the temporary storage unit 136.

the controller 68 may be configured to acquire information regarding the rotation of the EC supply pump 30d and the opening of the supply clamp 29B from the control circuit 32 of the culturing device 11 (step S3-3). Thereafter, the controller 68 may be configured to cause the upstream pump 142 to rotate for a predetermined period (same as the period for acquiring the sample from the reactor 12A) while closing the downstream clamp 146 (step S3-4). Thus, the sample from the reactor 12B may be stored in the temporary storage unit 136 in the same amount as the storage amount of the sample from the reactor 12A.

The controller 68 may be configured to acquire information regarding the rotation of the EC supply pump 30d and the opening of the supply clamp 29C from the control circuit 32 of the culturing device 11 (step S3-5). Thereafter, the controller 68 may be configured to cause the upstream pump 142 to rotate for a predetermined period (same as the period for acquiring the sample from the reactor 12A) while closing the downstream clamp 146 (step S3-6). Thus, the sample from the reactor 12C may be stored in the temporary storage unit 136 in the same amount as the storage amount of the sample from the reactor 12A.

The controller 68 may be configured to acquire information regarding the rotation of the EC supply pump 30d and the opening of the supply clamp 29D from the control circuit 32 of the culturing device 11 (step S3-7). Thereafter, the controller 68 may be configured to cause the upstream pump 142 to rotate for a predetermined period (same as the period for acquiring the sample from the reactor 12A) while closing the downstream clamp 146 (step S3-8). Thus, the sample from the reactor 12D may be stored in the temporary storage unit 136 in the same amount as the storage amount of the sample from the reactor 12A.

The controller 68 may be configured to acquire information regarding the rotation of the EC supply pump 30d and the opening of the supply clamp 29E from the control circuit 32 of the culturing device 11 (step S3-9). Thereafter, the controller 68 may be configured to cause the upstream pump 142 to rotate for a predetermined period (same as the period for acquiring the sample from the reactor 12A) while closing the downstream clamp 146 (step S3-10). Thus, the sample from the reactor 12E may be stored in the temporary storage unit 136 in the same amount as the storage amount of the sample from the reactor 12A.

By performing the above steps S3-1 to S3-10, a combined sample obtained by combining the samples from the reactors 12A to 12E may be stored in the temporary storage unit 136. The combined sample may be obtained by combining the same amount of the samples from the reactors 12A to 12E. Therefore, in the combined sample, the samples in one culturing device 11 may be averaged, and thus, the combined sample may be considered to indicate the culture state of the culturing device 11.

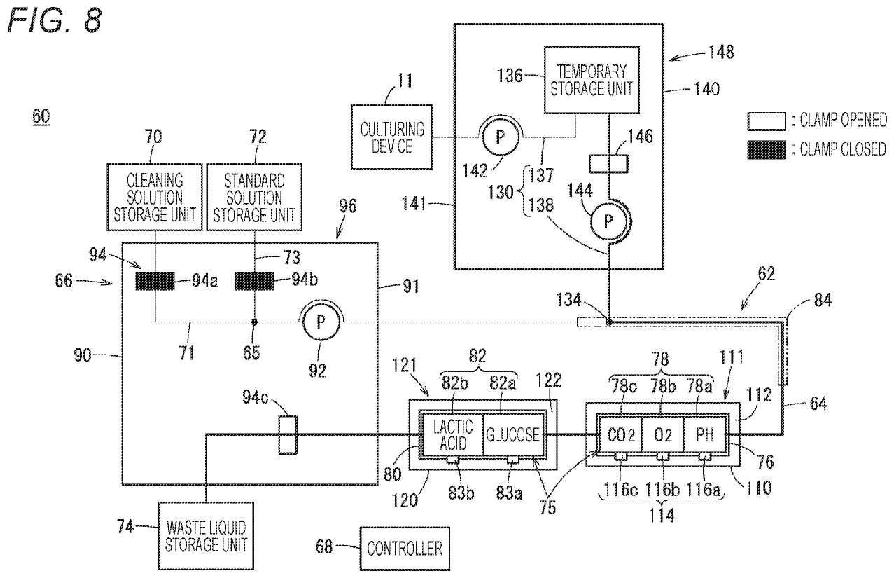

After the temporary storage step, the sampling device 60 may be configured to supply the combined sample in the temporary storage unit 136 to the sampling channel 64 in the next combined sample outflow step. During this process, the controller 68 may be configured to cause the downstream clamp 146 to open and to rotate the introduction pump 144 (step S3-11). During this process, the controller 68 may also be configured to cause the operation of the upstream pump 142 to stop. The controller 68 may be configured to cause the cleaning solution clamp 94a and the standard solution clamp 94b to close, to cause the waste liquid clamp 94c to open, and to cause rotation of the main-mechanism-side pump 92 and the upstream pump 142 to stop, as illustrated in FIG. 8. With the rotation of the introduction pump 144, the combined sample in the temporary storage unit 136 may flow out to the downstream line 138 of the sample introduction channel 130 at a flow rate of, for example, about 10 mL/min. As a result, the combined sample may flow into the connection part 84 (connection point 134) of the sampling channel 64 from the downstream line 138, sequentially flowing pass the first detection unit 76 and the second detection unit 80 and may be discharged to the waste liquid storage unit 74.

When the combined sample passes, the plurality of first elements 78 (e.g., pH chip 78a, O₂ chip 78b, and/or CO₂ chip 78c) of the first detection unit 76 may come into contact with the combined sample and may be colored according to the pH and/or the contents of O₂ and/or CO₂. The first measuring instrument 110 may be configured to optically measure each of the first elements 78 and to transmits the detection result to the controller 68. The controller 68 may be configured to receive the detection result and to perform appropriate processing to display the measured values (e.g., pH value, O₂ concentration, and/or CO₂ concentration) on the monitor 100 of the main mechanism 90.

Similarly, when the combined sample passes, the plurality of second elements 82 (e.g., glucose chip 82a and/or lactic acid chip 82b) of the second detection unit 80 may come into contact with the combined sample. The second measuring instrument 120 may be configured to detect current values corresponding to the contents of glucose and/or lactic acid. The second measuring instrument 120 may be configured to transmit each detection result to the controller 68. The controller 68 may be configured to receive detection result and to perform appropriate processing to display the measured values (e.g., glucose concentration and/or lactic acid concentration) on the monitor 100.

After the sampling step, the controller 68 may be configured to determine whether or not the cell culture of the culturing device 11 is completed (step S4 in FIG. 5). When the cell culture is not completed (step S4: NO), the cleaning step (step S5 in FIG. 5) may be performed. In the cleaning step, the controller 68 may be configured to cause the cleaning solution storage unit 70 to supply cleaning solution to the sampling channel 64 so as to remove the combined sample attached to the plurality of first elements 78 and the plurality of second elements 82.

The sampling device 60 may be configured to perform a calibration step (step S6 in FIG. 5), as necessary. In the calibration step, the controller 68 may be configured to cause the standard solution storage unit 72 to supply the standard solution to the sampling channel 64 to calibrate the second sensor unit 121 (second measuring instrument 120). In addition, the user may set the first measuring instrument 110 in the calibration device 118 (see FIG. 1) to calibrate the first measuring instrument 110.

When the cleaning step (or the calibration step) is completed, the controller 68 may be configured to return to step S3 and to perform the subsequent steps. When determining in step S4 that the cell culture is completed (step S4: YES), the controller 68 may be configured to end the operation flow of the sampling device 60.

It should be appreciated that the sampling device 60 is not necessarily limited to the above detailed configuration and that various methods of use may be adopted. Some other embodiments of the sampling device 60 are described below.

In at least one example embodiment, a sampling device 60A may be the same as sampling device 60 detailed above, but the sampling device 60A may be configured to store the sample from each of the reactors 12 in the temporary storage unit 136 without acquiring information regarding the opening of each of the supply clamps 29A to 29E from the culturing device 11.

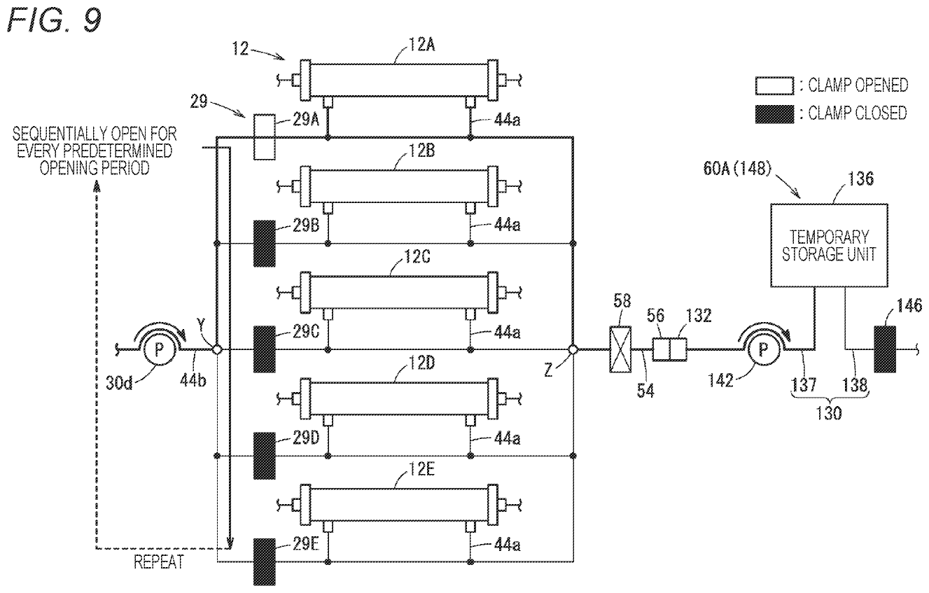

For example, the culturing device 11 may be configured to perform an operation of opening any one of the supply clamps 29A to 29E and closing the other four in the order of the reactors 12A to 12E for the same period while rotating the EC supply pump 30d as illustrated in FIG. 9. Thus, the culturing device 11 may sequentially and intermittently supply the culture medium to the EC circulation circuit 44a of each of the reactors 12A to 12E. In addition, the culturing device 11 may be configured to perform an operation of opening any one of the supply clamps of the IC supply circuits 42b and closing the other four for the same period in the order of the reactors 12A to 12E while rotating the IC supply pump 30b. Thus, the culturing device 11 may sequentially and intermittently supply the culture medium to the EC circulation circuit 44a of each of the reactors 12A to 12E.

In contrast, the controller 68 of the sampling device 60 may be configured to cause the upstream pump 142 to operate only for a period (hereinafter referred to as one cycle period) obtained by adding the opening periods of all the supply clamps 29A to 29E. For example, when the opening time of each of the supply clamps 29A to 29E of the culturing device 11 is about 6 seconds, the culture medium may be supplied from the EC supply circuit 44b to each of the reactors 12A to 12E (EC circulation circuits 44a) once in about 30 seconds. The controller 68 may be configured to set the time (e.g., about 30 seconds) of one cycle in which each of the supply clamps 29A to 29E is opened once as one cycle period. Then, in a culturing step for supplying and circulating the culture medium of the culturing device 11, the controller 68 may be configured to cause the upstream pump 142 to rotate for one cycle period at an appropriate timing at which sampling of the culture medium is considered to be necessary. The flow rate of each sample due to the rotation of the upstream pump 142 may be set to a value at which a combined sample in a target storage amount can be stored in the temporary storage unit 136. As an example, in a case where one cycle period is about 30 seconds and about 5 mL of the combined sample is to be stored in the temporary storage unit 136, the upstream pump 142 may be rotated so that the flow rate is about 10 mL/min.

The sampling device 60 can collect the same amount of samples from the reactors 12A to 12E by operating the upstream pump 142 for one cycle period, even if the rotation start timing of the upstream pump 142 does not coincide with the opening timing of each of the supply clamps 29A to 29E. For example, even if the rotation start timing of the upstream pump 142 is later than the opening timing of the supply clamp 29A by about 3 seconds, the supply clamp 29A may be opened in the latter half of one cycle period, so that the samples can be stored in the temporary storage unit 136 only during the opening period in which each of the supply clamps 29A to 29E is opened once as a whole.

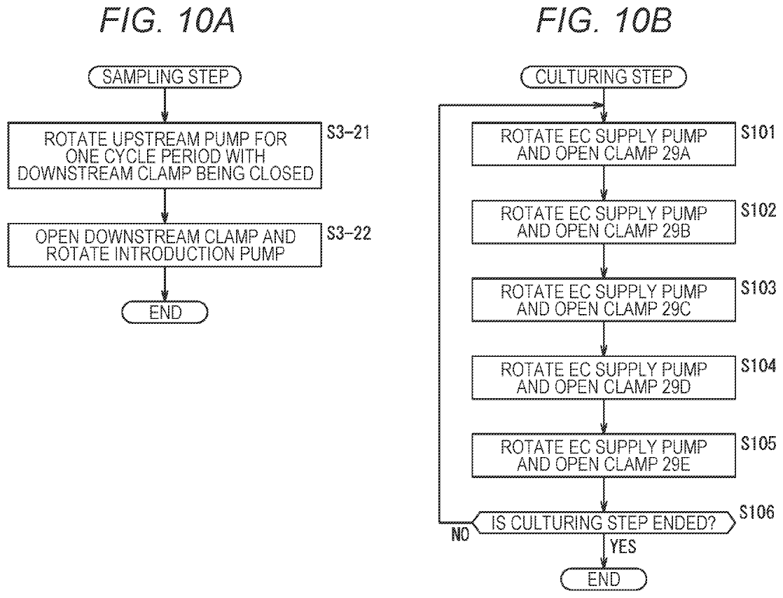

The sampling device 60A may be configured to perform a processing flow illustrated in FIG. 10A in the sampling step (step S3 in FIG. 5). That is, when starting the temporary storage step, the controller 68 may be configured to cause the upstream pump 142 to rotate for one cycle period while closing the downstream clamp 146 (step S3-21). It should be appreciated that the sampling device 60A is not limited to rotate the upstream pump 142 only for one cycle period and may, in at least one example embodiment, continuously or intermittently execute one cycle period twice or more (a times or more: a is a natural number). Thus, the sampling device 60A can store a sufficient amount of the combined sample in the temporary storage unit 136 even when the supply rate of the culture medium to each of the reactors 12A to 12E is low, for example.

As illustrated in FIG. 10B, during the temporary storage step, the culturing device 11 (control circuit 32) may be configured to cause the supply clamp 29A to open for a predetermined opening period while rotating the EC supply pump 30d and to cause the other supply clamps 29B to 29E to close for the same period as the opening period (step S101). With this process, the culture medium may be supplied to the EC circulation circuit 44a of the reactor 12A. Similarly, the culturing device 11 may be configured to cause the supply clamp 29B to open for a predetermined opening period while rotating the EC supply pump 30d and to cause the other supply clamps 29A and 29C to 29E to close for the same period as the opening period (step S102). In addition, the culturing device 11 may be configured to cause the supply clamp 29C to open for a predetermined opening period while rotating the EC supply pump 30d and to cause the other supply clamps 29A, 29B, 29D, and 29E to close for the same period as the opening period (step S103). In addition, the culturing device 11 may be configured to cause the supply clamp 29D to open for a predetermined opening period while rotating the EC supply pump 30d and to cause the other supply clamps 29A to 29C and 29E to close for the same period as the opening period (step S104). In addition, the culturing device 11 may be configured to cause the supply clamp 29E to open for a predetermined opening period while rotating the EC supply pump 30d and to cause the other supply clamps 29A to 29D to close for the same period as the opening period (step S105). With this process, the culture medium may be sequentially supplied to the EC circulation circuits 44a of the reactors 12B to 12E. Finally, the culturing device 11 may be configured to determine whether or not the culturing step is ended (step S106) and, when continuing the culturing step (step S106: NO), to return to step S101 and to repeat the same flow. When determining that the culturing step is ended (step S106: YES), the culturing device 11 may be configured to perform the next step (collection step of collecting cells in each of the reactors 12A to 12E).

As illustrated in FIG. 9, the sampling device 60A may be configured to move samples of the same amounts from the reactors 12A to 12E and into the temporary storage unit 136 by rotating the upstream pump 142 for one cycle period in which the culture medium is supplied to each of the reactors 12A to 12E. That is, a combined sample in which the samples are averaged may be stored in the temporary storage unit 136.

After the temporary storage step, the controller 68 may be configured to cause the downstream clamp 146 to open and to cause the introduction pump 144 to rotate (step S3-22). The controller 68 may be configured to cause the cleaning solution clamp 94a and the standard solution clamp 94b to close, to cause the waste liquid clamp 94c to open, and to cause rotation of the main-mechanism-side pump 92 and the upstream pump 142 to stop. As a result, the combined sample in the temporary storage unit 136 may flow into the downstream line 138 of the sample introduction channel 130, to sequentially flows through the connection part 84 of the sampling channel 64 and pass the first detection unit 76 and the second detection unit 80, and to the waste liquid storage unit 74 for discharge.

By operating the upstream pump 142 for one cycle period, the sampling device 60A may be configured to store the combined sample in which the samples are averaged in the temporary storage unit 136 without acquiring the information regarding the opening of each of the supply clamps 29A to 29E from the culturing device 11. The opening period of the supply clamps 29A to 29E by the culturing device 11 does not change. Therefore, periodic sampling can be performed a plurality of times during the culturing step by the culturing device 11 by initially setting one cycle period using the controller 68 of the sampling device 60.

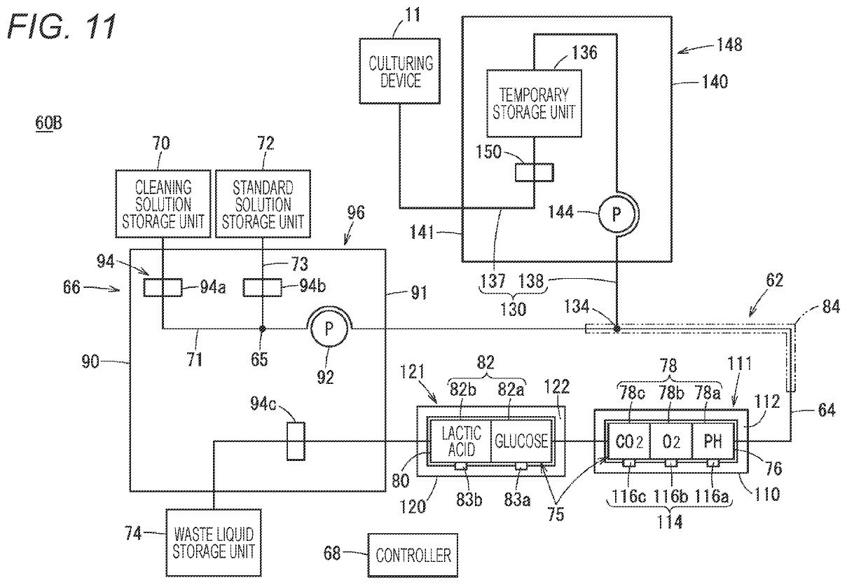

A sampling device 60B according to a second modification may be different from the sampling devices 60 and 60A in that the sampling device 60B may have an upstream clamp 150 in the upstream line 137 without having the upstream pump 142 and the downstream clamp 146, for example, as illustrated in FIG. 11. Similar to the sampling devices 60 and 60A described above, the introduction pump 144 may be provided on the downstream line 138.

A flexible medical bag that expands due to inflow of the sample and deflates (is flattened) due to outflow of the sample may be applied to the temporary storage unit 136. One end of the upstream line 137 may be connected to the lower side of the temporary storage unit 136 held by the main unit 96 or the introduction unit 148 in the direction of gravity. One end of the downstream line 138 may be connected to the upper side of the temporary storage unit 136 held by the main unit 96 or the introduction unit 148 in the direction of gravity.

The sampling device 60B may be configured to cause the introduction pump 144 to rotate while opening the upstream clamp 150, thereby applying a negative pressure to the upstream line 137 in the temporary storage step. That is, similar to the sampling device 60A, the controller 68 may be configured to cause the introduction pump 144 to rotate for one cycle period to allow the sample in each of the reactors 12A to 12E to flow into the temporary storage unit 136, and thus, can store the combined sample.

In the combined sample outflow step, the sampling device 60B may be configured to cause a negative pressure to be applied to the temporary storage unit 136 and the downstream line 138, for example, by rotating the introduction pump 144 with the upstream clamp 150 being closed. As a result, the combined sample in the temporary storage unit 136 may sequentially flows through the connection part 84 of the sampling channel 64 passing by the first detection unit 76 and the second detection unit 80 and to the waste liquid storage unit 74 to be discharged.

The sampling device 60B may be configured to store the sample in each of the reactors 12A to 12E in the temporary storage unit 136 under the operation of the introduction pump 144 even when the upstream pump 142 is not provided. Thus, in at least one example embodiment, the configuration of the introduction unit 148 may be simplified to achieve cost reduction and the introduction unit 148 may be more easily handled.

The sampling device 60B may also allow the sample of the culturing device 11 to flow into the temporary storage unit 136 without relying on the operation of the introduction pump 144. Specifically, the culturing device 11 may be configured to cause the clamps 28 provided in the IC waste liquid circuit 46 and the EC waste liquid circuit 48, respectively (see FIG. 2), to close. Thus, the culture medium (waste liquid) in each of the reactors 12A to 12E may flow out from the EC circulation circuit 44a to the sample introduction channel 130. That is, the sampling device 60B may be configured to store the combined sample in the temporary storage unit 136 by opening the upstream clamp 150 with the introduction pump 144 being stopped. At this time, since the flow rate of the sample flowing out to the sample introduction channel 130 may depend on the rotation speed of the EC supply pump 30d, the sampling device 60B may only need to appropriately control the opening period of the upstream clamp 150 according to the rotation speed of the EC supply pump 30d.

In at least one example embodiment, the present disclosure provides a sampling device 60, 60A, 60B for collecting a sample in a liquid form from a cell culturing device 11 that has a plurality of reactors 12 on the basis of a flow of a culture medium. The sampling device may include a sampling channel 64 through which the sample flows, a detection unit 75 provided in the sampling channel 64, a sample introduction channel 130 that connect to the sampling channel 64 upstream of the detection unit 75 and the culturing device 11, a pump (upstream pump 142, introduction pump 144) that allows the sample to flow through the sample introduction channel 130, and a control unit (controller 68) that is configured to operate, for example, the pump. The sampling device may also include a temporary storage unit 136 that may be provided in the sample introduction channel 130 and that may be capable of temporarily storing the sample. The temporary storage unit 136 may be configured to receive flow of the sample for each of the plurality of reactors 12 under the operation of the pump so as to store a combined sample that is obtained by combining a plurality of the samples and to send the combined sample to the sampling channel 64.

With this configuration, the sampling devices 60, 60A, and 60B may collectively store samples from the plurality of reactors 12 in the temporary storage unit 136 and may send the combined sample to the sampling channel 64, so that the sample in each of the plurality of reactors 12 can be more efficiently detected. As a result, the sampling devices 60, 60A, and 60B can satisfactorily monitor the culture state of the entire culturing device 11 (all of the plurality of reactors 12) while reducing the number of times of sampling and the sample amount.

The culturing device 11 may include a plurality of supply channels (EC supply circuits 44b) configured to supply a culture medium. The plurality of supply channels may be respectively connected to the plurality of reactors 12. The culturing device 11 may include a plurality of supply clamps 29 configured to open and close the plurality of supply channels, respectively. The culturing device 11 may include a control unit (controller 68). In at least one example embodiment, the control unit may be configured to operate the pump (upstream pump 142, introduction pump 144) for a predetermined period on the basis of acquisition of information regarding opening of any one of the plurality of supply clamps 29 from the culturing device 11. With this configuration, the sampling device 60 may be configured to store samples from the plurality of reactors 12 that are of a same amount in the temporary storage unit 136 and can more reliably obtain a combined sample in which the samples are averaged.

The culturing device 11 may include a plurality of supply channels (EC supply circuits 44b) configured to supply a culture medium. The plurality of supply channels may be respectively connected to the plurality of reactors 12. The culturing device 11 may include a plurality of supply clamps 29 configured to open and close the plurality of supply channels, respectively. The culturing device 11 may include a control unit (controller 68). In at least one example embodiment, the control unit may be configured to operate the pump (upstream pump 142, introduction pump 144) for one cycle period obtained by adding opening periods of all of the plurality of supply clamps 29. With this configuration, the sampling device 60A can easily obtain a combined sample in which the samples are averaged without necessarily acquiring information regarding opening of the supply clamp 29 from the culturing device 11.

The control unit (controller 68) may be configured to perform one cycle period for operating the pump (upstream pump 142, introduction pump 144) twice or more. With this configuration, the sampling device 60A can store a sufficient amount of the combined sample in the temporary storage unit 136.

In at least one example embodiment, the pump may be an upstream pump 142 provided in the sample introduction channel 130 between the culturing device 11 and the temporary storage unit 136. The control unit (controller 68) may be configured to operate the upstream pump 142 in a temporary storage step for allowing the sample in each of the plurality of reactors 12 to flow into the temporary storage unit 136 and to stops the operation of the upstream pump 142 in a combined sample outflow step for sending the combined sample from the temporary storage unit 136 to the sampling channel 64. With this configuration, the sampling device 60 can stably store the sample in each of the plurality of reactors 12 into the temporary storage unit 136 in the temporary storage step.

The sample introduction channel 130 may include a downstream clamp 146 that is configured to open and close the sample introduction channel 130 disposed between the sampling channel 64 and the temporary storage unit 136. The control unit (controller 68) may be configured to cause the downstream clamp 146 to close in the temporary storage step and to cause the downstream clamp 146 to open in the combined sample outflow step. With this configuration, the sampling devices 60 and 60A can prevent the sample from flowing out of the temporary storage unit 136 in the temporary storage step.

The sample introduction channel 130 may include an introduction pump 144 disposed between the sampling channel 64 and the temporary storage unit 136. The control unit (controller 68) may be configured to cause operation of the introduction pump 144 to stop in the temporary storage step and to operate the introduction pump 144 in the combined sample outflow step to send the combined sample to the sampling channel 64. With this configuration, the sampling devices 60 and 60A can smoothly introduce the combined sample into the sampling channel 64 in the combined sample outflow step.

The pump may include an introduction pump 144 provided in the sample introduction channel 130 between the sampling channel 64 and the temporary storage unit 136. the control unit (controller 68) may be configured to operate the introduction pump 144 in a temporary storage step to allow the sample in each of the plurality of reactors 12 to flow into the temporary storage unit 136 and to stop operation of the introduction pump 144 in a combined sample outflow step for sending the combined sample from the temporary storage unit 136 to the sampling channel 64. With this configuration, the sampling device 60B can guide the sample in each of the plurality of reactors 12 to the temporary storage unit 136 by applying a negative pressure to the sample introduction channel 130 on the upstream side of the introduction pump 144 under the operation of the introduction pump 144 in the temporary storage step. The sampling device 60B can also achieve cost reduction by eliminating the upstream pump 142.

The sample introduction channel 130 may include an upstream clamp 150 that is configured to open and close the sample introduction channel 130 between the culturing device 11 and the temporary storage unit 136. The control unit (controller 68) may be configured to cause the upstream clamp 150 to open in the temporary storage step and to cause the upstream clamp 150 to close in the combined sample outflow step. With this configuration, the sampling device 60B can store the samples from each of the plurality of reactors 12 in the temporary storage unit 136 in the temporary storage step and can introduce the combined sample to the sampling channel 64 in the combined sample outflow step.

In at least one example embodiment, the present disclosure provides a cell culturing system 10 for collecting a liquid sample from a cell culturing unit (culturing device 11) that includes a plurality of reactors 12 on the basis of a flow of a culture medium. The cell culturing unit may be configured to sequentially supply the culture medium to the plurality of reactors 12. The cell culturing system may include a sampling channel 64 through which the sample flows, a detection unit 75 provided in the sampling channel 64, a sample introduction channel 130 that connects the sampling channel 64 upstream of the detection unit 75 and the culturing unit, a pump (upstream pump 142, introduction pump 144) that allows the sample to flow through the sample introduction channel 130, and a control unit (controller 68) that is configured to operate the pump. The cell culturing system may also include a temporary storage unit 136 provided in the sample introduction channel 130 and that is capable of temporarily storing the sample. The temporary storage unit 136 may be configured to receive flow of the sample for each of the plurality of reactors 12 under the operation of the pump to store a combined sample obtained by combining a plurality of the samples and to send the combined sample to the sampling channel 64. With this configuration, the cell culturing system 10 can more efficiently detect a sample for each of the plurality of reactors 12.

1. A sampling device that receives two or more samples from a cell culturing device, the sampling device comprising:
a sample introduction channel including
a pump that is configured to move the two or more samples through the sample introduction channel, and
a temporary storage unit that is configured to receive a first sample of the two or more samples from a first reactor of a plurality of reactors of the cell culturing device and a second sample of the two or more samples from a second reactor of the plurality of reactors, to form a combined sample including at least a portion of each of the two or more samples, and to store the first sample, the second sample, or the combined sample.

2. The sampling device of claim 1, wherein the sampling device further includes a sampling channel, the sample introduction channel connecting the sampling channel and the cell culturing device, the sampling channel receiving the combined sample from the temporary storage unit.

3. The sampling device of claim 2, wherein the sampling device further includes a detection unit provided in the sampling channel for measuring properties of the combined sample, the introduction channel connected to the sampling channel upstream of the detection unit.

4. The sampling device according to claim 1, wherein the pump is provided between the culturing device and the temporary storage unit.

5. The sampling device of claim 1, wherein the sampling device further includes a control unit that is configured to operate the pump.

6. The sampling device of claim 5, wherein the cell culturing device further includes a plurality of supply channels for supplying a culture medium to the plurality of reactors, and a plurality of supply clamps for opening and closing the plurality of supply channels, and
wherein the control unit is configured to receive information from the cell culturing device regarding the openings and closings of the different supply clamps of the plurality of supply clamps, and the control unit operates the pump in response to the information.

7. The sampling device of claim 6, wherein the control unit operates the pump for a predetermined period.

8. The sampling device of claim 6, wherein the control unit operates the pump for one cycle period, the one cycle period determined by adding together opening periods of each of the plurality of supply clamps.

9. The sampling device according to claim 1, wherein the control unit has a first phase that causes the pump to run moving the two or more samples from the plurality of reactors to the temporary storage unit and a second phase that causes the pump to stop the operation to move the combined sample from the temporary storage to the sampling channel.

10. The sampling device of claim 9, wherein the sample introduction channel further includes a clamp between the temporary storage unit and the sampling channel that is configured to open and close the sample introduction channel, the control unit configured to cause the downstream clamp to close in the first phase and to cause downstream clamp to open in the second phase.

11. The sampling device of claim 9, wherein the pump is a first pump between the culturing device and the temporary storage unit, and the sample introduction channel further includes a second pump between the temporary storage unit and the sampling channel.

12. The sampling device of claim 11, wherein the control unit is configured to cause operations of the second pump to stop in the first phase and to cause operations of the second pump to run in the second phase.

13. The sampling device of claim 9, wherein the sample introduction channel further includes an upstream clamp between the culturing device and the temporary storage unit that is configured to open and close the sample introduction channel, the control unit configured to cause the clamp to open in the first phase and to cause the clamp to open in the second phase.

14. A cell culturing system comprising:
a cell culturing device having a plurality of reactors;
a sampling device having a sampling channel that includes a detection unit for measuring properties of a sample as received from the cell culturing device, the sample including at least portions of subsamples from two or more reactors of the plurality of reactors; and
a sample introduction channel fluidically connecting the cell culturing device and the sampling device, the sampling introduction channel fluidically connected to the sampling device upstream of the detection unit, the sampling introduction channel including:
a pump that is configured to cause the sample to flow from the cell culturing device through the sample introduction channel and to the sampling device, and
a temporary storage unit that is configured to receive the subsamples from the two or more reactors of the plurality of reactors, to form the sample by combining the portions of the subsamples from the two or more reactors, and to temporarily store the subsamples or sample before movement of the sample to the sampling device.

15. The cell culturing system of claim 14, wherein the pump is a first pump between the cell culturing device and the temporary storage unit, and the sample introduction channel further includes a second pump between the temporary storage unit and the sampling device.

16. The cell culturing system of claim 14, wherein the sample introduction channel further includes a clamp between the cell culturing device and the temporary storage unit.

17. The cell culturing system of claim 16, wherein the clamp is a first clamp, and the sample introduction channel further includes a second clamp between the temporary storage unit and the sampling device.

18. The cell culturing system of claim 14, wherein the cell culturing system further includes a control unit that is configured to operate the pump.

19. The cell culturing system of claim 18, wherein the cell culturing device further includes a plurality of supply channels for supplying a culture medium to the plurality of reactors, and a plurality of supply clamps for opening and closing the plurality of supply channels, and
wherein the control unit is configured to receive information from the cell culturing device regarding the openings and closings of the different supply clamps of the plurality of supply clamps, and the control unit operates the pump in response to the information.

20. A sampling device that receives a sample from a cell culturing device, the sampling device comprising:
a sampling device having a sampling channel that includes a detection unit for measuring properties of the sample as received from the cell culturing device, the sample including at least portions of subsamples from two or more reactors of a plurality of reactors of the cell culturing device; and
a sample introduction channel that fluidically connects the sampling device and the cell culturing device, the sample introduction channel including
a pump that is configured to cause the sample to flow from the cell culturing device through the sample introduction channel and to the sampling device, and
a temporary storage unit that is configured to receive the subsamples from the two or more reactors of the plurality of reactors, to form the sample by combining the portions of subsamples from the two or more reactors, and to temporarily store the subsamples or sample before movement of the sample to the sampling device.
